(19) United States
(12) Patent Application Publication
(10) Pub. No.: US 2023/0256058 A1
(43) Pub. Date: Aug. 17, 2023

(54) LOW BURST-RELEASE LIRAGLUTIDE DEPOT SYSTEMS

(71) Applicant: MAPI PHARMA LTD., Ness Ziona (IL)

(72) Inventors: Galina ZATS, Rehovot (IL); Nadav BLEICH KIMELMAN, Tel Aviv (IL); Shai RUBNOV, Tel Aviv (IL); Ehud MAROM, Tel Aviv (IL)

(73) Assignee: MAPI PHARMA LTD., Ness Ziona (IL)

(21) Appl. No.: 18/001,610

(22) PCT Filed: Apr. 26, 2021
(86) PCT No.: PCT/IL2021/050479

§ 371 Date: Dec. 13, 2022

Related U.S. Application Data
(60) Provisional application No. 63/045,213, filed on Jun. 29, 2020.

(51) Int. Cl. A61K 38/26; A61K 9/50; A61K 9/48; A61P 3/04; A61P 3/10

(52) U.S. Cl. CPC A61K 38/26; A61K 9/5073; A61K 9/4866; A61P 3/04; A61P 3/10

(57) ABSTRACT

The present invention provides parenteral pharmaceutical compositions comprising therapeutically effective amounts of liraglutide or pharmaceutically acceptable salts thereof, the parenteral pharmaceutical compositions are formulated in depot form and provide low-burst release in the first twenty-four hours post administration. The present invention further provides methods of use of the parenteral pharmaceutical compositions for treating type-2 diabetes mellitus and Parkinson's disease.

### FIELD OF THE INVENTION

The present invention relates to parenteral pharmaceutical compositions comprising glucagon-like peptide-1 (GLP-1) receptor agonists and use thereof for treating type-2 diabetes mellitus and Parkinson's Disease. Particularly, the present invention relates to low-burst liraglutide depot systems for sustained release administration, useful in treating type-2 diabetes mellitus and Parkinson's Disease.

### BACKGROUND OF THE INVENTION

Despite advances in the treatment of type-2 diabetes, optimal glycemic control is often not achieved. Hypoglycemia and weight gain associated with many antidiabetic medications may interfere with the implementation and long-term application of intensive therapies. Current treatments have centered on increasing insulin availability (either through direct insulin administration or through agents that promote insulin secretion), improving sensitivity to insulin, delaying the delivery and absorption of carbohydrates from the gastrointestinal tract, or increasing urinary glucose excretion.

Diabetes mellitus type II or type-2 diabetes (formerly called non-insulin-dependent diabetes mellitus (NIDDM), or adult-onset diabetes) is a disorder that is characterized by high blood glucose levels associated with insulin resistance and relative insulin deficiency. While it is often initially managed by increasing exercise and dietary modifications, medications are typically needed as the disease progresses. In the United States, more than thirty million people of all ages (9.4% of the population) have been diagnosed in the year of 2015 as having diabetes. About 90% to 95% of the subjects who suffer from diabetes are diagnosed as having type-2 diabetes mellitus (National Diabetes Statistics Report, 2017). Type-2 diabetes mellitus is characterized by insulin resistance and by some impairment in insulin secretion leading to hyperglycemia. During periods of hyperglycemia, there is an increase in glucose transport into β cells of the pancreas, which leads to insulin secretion. Insulin secretion occurs in two phases. The first phase occurs after a meal and is manifested by an immediate rise in insulin secretion, which lasts approximately 10 minutes. Following the first phase, the second phase occurs which is manifested by a slow release of insulin for a prolonged period of time. Patients with type-2 diabetes have markedly reduced first phase insulin secretion, leading to persistently elevated postprandial glucose concentrations despite relatively normal fasting glucose levels.

Glucagon-like peptide-1 (GLP-1) is a natural peptide of 30 amino acid residues which is secreted by intestinal cells after meals. It stimulates glucose-dependent insulin release and suppresses postprandial glucagon secretion. GLP-1 is not highly efficacious when administered as a therapeutic agent due to its short pharmacokinetic half-life, i.e., approximately 2-5 min., mainly because it is sensitive to enzymatic degradation by the dipeptidyl-peptidase enzyme (DPP-4). In order to lengthen the half-life of GLP-1, metabolically stable GLP-1 analogs containing chemical modifications and amino acid substitutions, have been developed.

Liraglutide (VICTOZA®) is a GLP-1 receptor agonist. The peptide precursor of liraglutide, produced by a process that includes expression of recombinant DNA in Saccharomyces cerevisiae, has been engineered to be 97% homologous to native human GLP-1 by substituting arginine for lysine at position 34. Liraglutide is produced by attaching a C-16 fatty acid (palmitic acid) with a glutamic acid spacer on the remaining lysine residue at position 26 of the peptide precursor. In the United States, liraglutide is indicated (a) as an adjunct to diet and exercise to improve glycemic control in patients 10 years and older with type-2 diabetes mellitus; and (b) to reduce the risk of major adverse cardiovascular events in adults with type-2 diabetes mellitus and established cardiovascular disease. Mechanistically, liraglutide activates the GLP-1 receptor, increases intracellular cyclic AMP (cAMP) leading to insulin release in the presence of elevated glucose concentrations. Liraglutide also decreases glucagon secretion in a glucose-dependent manner. Liraglutide is administered by subcutaneous injections. Adult dosage includes a 0.6 mg daily initiation dose for one week, then an increase to 1.2 mg daily. If additional glycemic control is required, the dose is increased to 1.8 mg daily. The human range for clinically effective concentrations of liraglutide is 5-45 nmol/L (Ingwersen S. H. et al. J. Clin. Pharmacol. 2012; 52:1815-1823). The steady state therapeutic plasma concentration is about 128 ng/mL (VICTOZA® Prescribing Information).

Glucagon-like peptide-1 (GLP-1) receptor agonists also have neuroprotective effects in preclinical models of Parkinson's disease (PD). Liraglutide can reduce systemic and brain insulin resistance, an abnormality that could drive Parkinson's pathogenesis. Indeed, impaired insulin signaling in the brain can cause or exacerbate many brain pathologies and behavioral abnormalities seen in PD. Another GLP-1 agonist, exenatide, has shown to induce significant improvement of motor and cognitive symptoms in patients with Parkinson's disease (Athauda, D. et al. The Lancet, 2017, vol. 390, pp. 1664-1675).

WO 2017/025990 discloses a composition comprising particles, wherein said particles comprise poly(lactide-co-glycolide) polymer, therapeutically effective amount of liraglutide or a pharmaceutically acceptable salt thereof and a hydrophilic particle size modulating agent, wherein the composition is free of added divalent metal ions.

CN 102085355 discloses long-acting microsphere injection of liraglutide, PLGA, excipients and a surfactant. CN 102429876 discloses a sustained release microsphere preparation comprising liraglutide and a biodegradable substrate material selected from polylactic acid (PLA), polylactic acid-glycolic acid (PLGA) block copolymer, glycolide-lactide (PLCG) copolymer, polyglycolic acid (PGA), polycaprolactone (PCL), polylactic acid-glycolic acid-polycaprolactone (PLGA-PCL) copolymer or PLCG-polycaprolactone (PLCG-PCL). CN 104434817 discloses a sustained release microsphere preparation for an injection of liraglutide, comprising liraglutide, polylactic acid-glycolic acid copolymer (PLGA) and polyvinyl alcohol (PVA). CN 104825405 discloses liraglutide multi-emulsion microspheres comprising polylactic acid-glycolic acid copolymer (PLGA) and polyvinyl alcohol (PVA). CN 102688198 (WO 2013/189282) discloses a method for preparing polypeptide drug microsphere formulation using an oil-in-oil emulsion technique.

There remains an unmet need in the art for improved long-acting formulations of liraglutide that can provide safe and effective release of the active ingredient over an extended period of time.

### SUMMARY OF THE INVENTION

The present invention provides parenteral pharmaceutical compositions comprising therapeutically effective amounts of GLP-1 receptor agonists, in particular liraglutide, or pharmaceutically acceptable salts thereof, wherein the parenteral pharmaceutical compositions are formulated in a depot form. The present invention further provides methods of treating type-2 diabetes mellitus and Parkinson's disease comprising administering to a subject in need thereof the parenteral pharmaceutical compositions of the present invention.

The present invention is based in part on the surprising discovery that a depot formulation of liraglutide and a biodegradable carrier comprising, e.g., polylactides, polyglycolides and/or polycaprolactones, provides long-acting therapeutically effective plasma concentrations of the liraglutide active ingredient that are equivalent or superior to known injectable liraglutide compositions suitable for daily administration. Specifically, the depot formulations of the invention achieve at least one of the following attributes: i) plasma concentrations of liraglutide of at least about 5 nmol/L for about 1 week to about 28 days after a single administration; and ii) a twenty-four hour liraglutide burst release of less than 15% of the administered dose following administration. The formulations of the present invention are effective in reducing HbA1C and plasma glucose levels and provide equivalent or superior plasma concentrations after a single administration over a period of about 1 week or longer as compared to daily injections of conventional liraglutide formulations. It is contemplated that the formulations disclosed herein provide equal or superior therapeutic efficacy to the commercially available daily injectable dosage forms of liraglutide, with reduced incidence of side effects and/or with reduced severity of side effects. By comparison to formulations derived from other sustained release drug delivery technologies, the liraglutide sustained release composition of the present invention provides a superior release kinetics with minimal burst, increased duration of drug release with less frequent injections, and improved local tissue tolerance due to a small injection volume.

Thus, in one aspect, the present invention provides a long-acting parenteral pharmaceutical composition comprising microparticles comprising dried water-in-oil-in-water (w/o/w) double emulsion droplets comprising an internal aqueous phase comprising a therapeutically effective amount of liraglutide or a pharmaceutically acceptable salt thereof; a water immiscible polymeric phase comprising a biodegradable carrier selected from the group consisting of polylactides, polyglycolides, polycaprolactones, and combinations thereof; and an external aqueous phase, wherein the composition is in depot form suitable for implantation at a medically acceptable location in a subject in need thereof at a frequency of once weekly to once every six months and provides a twenty-four hour liraglutide burst release of less than 15% of the administered dose following administration.

In one embodiment, the composition provides a twenty-four hour liraglutide burst release of less than 10% of the administered dose following administration. In another embodiment, the composition provides a twenty-four hour liraglutide burst release of less than 5% of the administered dose following administration.

In another aspect, the present invention provides a long-acting parenteral pharmaceutical composition comprising microparticles comprising dried water-in-oil-in-water (w/o/w) double emulsion droplets comprising an internal aqueous phase comprising a therapeutically effective amount of liraglutide or a pharmaceutically acceptable salt thereof; a water immiscible polymeric phase comprising a biodegradable carrier selected from the group consisting of polylactides, polyglycolides, polycaprolactones, and combinations thereof; and an external aqueous phase, wherein the composition is in depot form suitable for implantation at a medically acceptable location in a subject in need thereof at a frequency of once weekly to once every six months and provides average human steady-state plasma concentrations (C_{ss,avg}) of liraglutide of at least about 5 nmol/L for about 1 week to about 28 days after a single administration.

In one embodiment, the average human steady-state plasma concentrations (C_{ss,avg}) of liraglutide are between about 5 nmol/L and about 45 nmol/L, including each value within the specified range. In further embodiments, the average human steady-state plasma concentrations (C_{ss,avg}) of liraglutide of between about 5 nmol/L and about 45 nmol/L are achieved 2 days or more after a single parenteral administration.

In some embodiments, the present invention provides a method of achieving average human steady-state plasma concentrations (C_{ss,avg}) of liraglutide of at least about 5 nmol/L for about 1 week to about 28 days after a single administration, by administering to a subject in need thereof a long-acting parenteral pharmaceutical composition comprising liraglutide or a pharmaceutically acceptable salt thereof and a biodegradable carrier selected from the group consisting of polylactides, polyglycolides, polycaprolactones, and combinations thereof, wherein the composition is in depot form suitable for implantation at a medically acceptable location in a subject in need thereof.

In various embodiments, the present invention provides a method of reducing fasting glucose levels in a subject by at least about 20% for a time period between about 1 week and about 28 days after a single administration, by administering to a subject in need thereof a long-acting parenteral pharmaceutical composition comprising liraglutide or a pharmaceutically acceptable salt thereof and a biodegradable carrier selected from the group consisting of polylactides, polyglycolides, polycaprolactones, and combinations thereof, wherein the composition is in depot form suitable for implantation at a medically acceptable location in a subject in need thereof. In one embodiment, the fasting glucose levels are reduced by at least 30% for a time period between about 1 week and about 28 days after a single administration. In another embodiment, the fasting glucose levels are reduced by at least 30% up to 14 days after administration of the depot composition of the invention. In yet another embodiment, the fasting glucose levels after a single parenteral administration are between about 70 and about 400 mg/dL, including each value within the specified range.

In certain embodiments, the present invention provides a method of reducing fed glucose levels in a subject by at least about 10% for a time period between about 1 week and about 28 days after a single administration, by administering to a subject in need thereof a long-acting parenteral pharmaceutical composition comprising liraglutide or a pharmaceutically acceptable salt thereof and a biodegradable carrier selected from the group consisting of polylactides, polyglycolides, polycaprolactones, and combinations thereof, wherein the composition is in depot form suitable for implantation at a medically acceptable location in a subject in need thereof. In one embodiment, the fed glucose levels are reduced by at least 15% for a time period between about 1 week and about 28 days after a single administration. In another embodiment, the fed glucose levels are reduced by at least 20% up to 10 days after administration of the depot composition of the invention. In yet another embodiment, the fed glucose levels after a single parenteral administration are between about 120 and about 650 mg/dL, including each value within the specified range.

In other embodiments, the present invention provides a method of reducing hemoglobin A1C (HbA1C) levels in a subject by at least about 20% for a time period between about 1 week and about 28 days after a single administration, by administering to a subject in need thereof a long-acting parenteral pharmaceutical composition comprising liraglutide or a pharmaceutically acceptable salt thereof and a biodegradable carrier selected from the group consisting of polylactides, polyglycolides, polycaprolactones, and combinations thereof, wherein the composition is in depot form suitable for implantation at a medically acceptable location in a subject in need thereof. In one embodiment, the hemoglobin A1C (HbA1C) levels after a single parenteral administration are between about 4% and about 10.5%, including each value within the specified range.

In particular embodiments, liraglutide is present in the pharmaceutical composition in the form of a free base.

In various embodiments, the biodegradable carrier is a biodegradable polymer selected from the group consisting of poly (D,L-lactide-co-glycolide) (PLGA), poly (D,L-lactide) (PLA), polyglycolide (PGA), polycaprolactone (PCL), and combinations thereof. Each possibility represents a separate embodiment of the present invention. In one currently preferred embodiment, the biodegradable carrier is poly (D,L-lactide-co-glycolide) (PLGA). In another currently preferred embodiment, the biodegradable carrier is poly (D,L-lactide) (PLA). In yet another currently preferred embodiment, the biodegradable carrier is poly (D,L-lactide)-polycaprolactone (PLA-PCL). In an additional currently preferred embodiment, the biodegradable carrier is a mixture of poly (D,L-lactide-co-glycolide) and poly (D,L-lactide)-polycaprolactone (PLGA/PLA-PCL).

In some embodiments, the external aqueous phase comprises a surfactant selected from the group consisting of polyvinyl alcohol (PVA), polysorbate, polyethylene oxide-polypropylene oxide block copolymers, and cellulose esters. Each possibility represents a separate embodiment. In a currently preferred embodiment, the surfactant is PVA. In other embodiments, the external aqueous phase further comprises a tonicity modifier. In one currently preferred embodiment, the tonicity modifier comprises sodium chloride.

In some embodiments, the depot composition of the present invention is in the form of solid microparticles, a solution or a suspension. Each possibility represents a separate embodiment of the present invention. In currently preferred embodiments, the composition is in the form of a suspension comprising solid microparticles suspended in a physiologically acceptable solvent.

In certain embodiments, the depot composition of the present invention is prepared by a water-in-oil-in-water (w/o/w) double emulsification process comprising the steps of:
(i) dispersing an aqueous suspension or solution of liraglutide or a pharmaceutically acceptable salt thereof in a solution of the biodegradable carrier in a water-immiscible volatile organic solvent, thereby obtaining a water-in-oil emulsion; and
(ii) dispersing said water-in-oil emulsion in a continuous external water phase comprising a surfactant, to form a water-in-oil-in-water (w/o/w) double emulsion droplets.

In several embodiments, the process further comprises the step of (iii) collecting the thus formed microparticles by filtration or centrifugation. In other embodiments, the process further comprises the step of (iv) washing the collected microparticles with purified water. In further embodiments, the process further comprises the step of lyophilizing the collected microparticles. In additional embodiments, the process further comprises the step of reconstituting the lyophilized microparticles in a physiologically acceptable solvent prior to administration.

In further embodiments, the composition is suitable for a dosing schedule from about once weekly to about once every six months, including each value within the specified range. In other embodiments, the composition releases the liraglutide active ingredient over a period of about one month to about three months, including each value within the specified range. In additional embodiments, the composition releases the liraglutide active ingredient over a period of about one week to about one month, including each value within the specified range. In a currently preferred embodiment, the composition releases the liraglutide active ingredient over a period of about one week to about two weeks, including each value within the specified range.

In other embodiments, the liraglutide is administered at a dose between about 5 mg and about 100 mg, including each value within the specified range.

As contemplated herein, the compositions of the invention are useful in treating subjects afflicted with diabetes, in particular type-2 diabetes mellitus.

Thus, in some embodiments, the present invention provides a method of treating type-2 diabetes mellitus, comprising the step of administering to a subject in need thereof a parenteral pharmaceutical composition as disclosed herein. In one embodiment, said treatment comprises reducing fasting glucose levels in said subject by at least about 20%, preferably by at least about 30%. In another embodiment, said treatment comprises reducing fed glucose levels in said subject by at least about 10%, preferably by at least about 15%, and more preferably by at least about 20%. In yet another embodiment, said treatment comprises reducing hemoglobin A1C (HbA1C) levels in said subject by at least about 20%, preferably by at least about 30%. In particular embodiments, the dose sufficient to achieve said reduction in fasting glucose levels, fed glucose levels, or hemoglobin A1C (HbA1C) levels provides average human steady-state plasma concentrations (C_{ss,avg}) of liraglutide of at least about 5 nmol/L, preferably between about 5 nmol/L and about 45 nmol/L for about 1 week to about 28 days after a single administration.

According to additional embodiments, the compositions of the invention are useful in treating subjects afflicted with Parkinson's Disease. Thus, in some embodiments, the present invention relates to a method of treating Parkinson's Disease, comprising the step of administering to a subject in need thereof a parenteral pharmaceutical composition as disclosed herein.

In other embodiments, the present invention provides a long-acting parenteral pharmaceutical composition comprising liraglutide or a pharmaceutically acceptable salt thereof, a biodegradable carrier selected from the group consisting of polylactides, polyglycolides, polycaprolactones, and combinations thereof, and at least one divalent metal ion, wherein the composition is in depot form suitable for implantation at a medically acceptable location in a subject in need thereof. In some embodiments, the composition provides average human steady-state plasma concentrations (C_{ss,avg}) of liraglutide of at least 5 nmol/L for about 1 week to about 28 days after a single administration to a subject. In other embodiments, the composition provides average human steady-state plasma concentrations (C_{ss,avg}) of liraglutide of between about 5 nmol/L and about 45 nmol/L for about 1 week to about 28 days after a single administration to a subject. In yet other embodiments, the composition provides a twenty-four hour liraglutide burst release of less than 15% of the administered dose following administration. In additional embodiments, the composition provides a twenty-four hour liraglutide burst release of less than 10% of the administered dose following administration. In further embodiments, the composition provides a twenty-four hour liraglutide burst release of less than 5% of the administered dose following administration. In some embodiments, the at least one divalent metal ion is zinc.

In some embodiments, the composition comprises liraglutide, a biodegradable polymer selected from the group consisting of poly (D,L-lactide-co-glycolide) (PLGA), poly (D,L-lactide) (PLA), polyglycolide (PGA), polycaprolactone (PCL), and combinations thereof, and zinc oxide. In further embodiments, the biodegradable polymer is PLGA. In other embodiments, the biodegradable polymer is PLA-PCL. In yet other embodiments, the biodegradable polymer is PLGA/PLA-PCL. In some embodiments, the composition comprises zinc oxide. In other embodiments, the composition comprises poloxamer.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1: shows the effect of test formulations on fasting glucose levels (mg/dL): (a) Naïve (wild type, untreated) (n=6); (b) vehicle control (saline), 1 ml/kg, IM (n=8); (c) L-API, 200 mg/kg, SC, daily (n=9-10); (d) LIR-110919 (PLGA-liraglutide depot), 156.3 mg/kg, IM on Day-1 (n=10); (e) LIR-221019 (PCL-PLA/PLGA liraglutide depot) 266.7 mg/kg, IM on Day-1 (n=8-10). Data are shown as Mean+SEM, * p<0.05 against vehicle. One Way ANOVA followed by Dunnett's test.

FIG. 2: shows the effect of test formulations on ad lib fed glucose levels (mg/dL): (a) Naïve (wild type, untreated) (n=6); (b) vehicle control (saline), 1 ml/kg, IM (n=8); (c) L-API, 200 mg/kg, SC, daily (n=9-10); (d) LIR-110919 (PLGA-liraglutide depot), 156.3 mg/kg, IM on Day-1 (n=10); (e) LIR-221019 (PCL-PLA/PLGA liraglutide depot) 266.7 mg/kg, IM on Day-1 (n=8-10). Data are shown as Mean+SEM, * p<0.05 against vehicle. #p<0.05 against L-API. One Way ANOVA followed by Dunnett's test.

FIG. 3: shows the effect of test formulations on HbA1C (%). (a) Naïve (wild type, untreated) (n=6); (b) vehicle control (saline), 1 ml/kg, IM (n=8); (c) L-API, 200 mg/kg, SC, daily (n=9-10); (d) LIR-110919 (PLGA-liraglutide depot), 156.3 mg/kg, IM on Day-1 (n=10); (e) LIR-221019 (PCL-PLA/PLGA liraglutide depot) 266.7 mg/kg, IM on Day-1 (n=8-10). Data are shown as Mean+SEM, * p<0.05 against vehicle. One Way ANOVA followed by Dunnett's test.

FIG. 4: shows the effect of test formulations on body weight (g). (a) Naïve (wild type, untreated) (n=6); (b) vehicle control (saline), 1 ml/kg, IM (n=8); (c) L-API, 200 mg/kg, SC, daily (n=9); (d) LIR-110919 (PLGA-liraglutide depot), 156.3 mg/kg, IM on Day-1 (n=10); (e) LIR-221019 (PCL-PLA/PLGA liraglutide depot) 266.7 mg/kg, IM on Day-1 (n=8-10). Data are shown as Mean+SEM.

FIGS. 5A-5C: show the plasma concentrations of test formulations on feed intake in db/db mice in μg/mL. (5A) L-API, 200 mg/kg, SC, daily injections (n=9-10); (5B) LIR-110919 (PLGA-liraglutide depot), 156.3 mg/kg, IM on Day-1 (n=10); (5C) LIR-221019 (PCL-PLA/PLGA liraglutide depot) 266.7 mg/kg, IM on Day-1 (n=8-10). Data are shown as Mean+SEM.

### DETAILED DESCRIPTION OF THE INVENTION

The present invention provides long-acting pharmaceutical preparations of liraglutide or pharmaceutically acceptable salts thereof which afford equal or superior therapeutic efficacy to known commercial once a day compositions but are designed for administration at a frequency of once weekly to once every six months and thus result in improved patient compliance. In addition to providing the same or superior therapeutic efficacy, the pharmaceutical preparations of the invention reduce side effects (local and/or systemic), resulting from frequent injections of liraglutide including lipoatrophy, lipohypertrophy, local allergic reactions, abscess formation and scarring. The present invention further provides low-burst release liraglutide depot formulations that avoid the undesired release of high concentrations of liraglutide shortly after administration thus resulting in improved glycemic control and prevention of events of hypoglycemia within the first 24 hours following administration.

According to some aspects and embodiments, the pharmaceutical formulations and dosages of the invention are conveniently provided in a form suitable for parenteral administration, for example by injection, implantation or infusion. Each possibility represents a separate embodiment of the invention. The term “parenteral” as used herein refers to routs of administration selected from subcutaneous (SC), intravenous (IV), intramuscular (IM), intradermal (ID), intraperitoneal (IP), and the like. Each possibility represents a separate embodiment of the invention.

Within the scope of the present invention are sustained release depot formulations. The term “sustained” as used herein refers to a pharmaceutical composition which provides prolonged, long or extended release of a therapeutically effective amount of liraglutide or any other pharmaceutically acceptable salt thereof, to the general systemic circulation of a subject or to local sites of action in a subject. This term may further refer to a pharmaceutical composition which provides prolonged, long or extended exposure to (pharmacokinetics) and duration of action of (pharmacodynamics) a therapeutically effective amount of liraglutide or any other pharmaceutically acceptable salt thereof, in a subject. In particular, the sustained release pharmaceutical composition of the present invention provides a dosing regimen of once a week, twice a week, once every two weeks, once a month, once every two months, once every three months, once every four months, once every five months, or once every six months. Each possibility represents a separate embodiment of the invention.

According to further embodiments, the depot form is suitable for subcutaneous administration or intramuscular implantation. Each possibility represents a separate embodiment of the invention. Depending on the duration of action required, each depot or implantable device of the present invention, is designed to afford the release of liraglutide or a pharmaceutically acceptable salt thereof over a period selected from the group consisting of 2 days, 3 days, 4 days, 5 days, 6 days, a week, a week and a half, two weeks, three weeks, four weeks, a month, a month and a half, two months, two months and a half, three months, three months and a half, four months, four months and a half, five months, five months and a half, and six months. Each possibility represents a separate embodiment.

The depot system of the present invention encompasses any form known to a person of skill in the art. According to some embodiments, the depot system is present in a form selected from the group consisting of biodegradable microspheres, non-biodegradable microspheres, implants of any suitable geometric shape, prolonged release gels, and erodible matrices. Each possibility represents a separate embodiment. According to certain embodiments, the implant of any suitable geometric shape is selected from the group consisting of implantable capsules, implantable rods and implantable rings. Each possibility represents a separate embodiment of the invention.

According to some embodiments, a suitable form of parenteral pharmaceutical compositions includes, but is not limited to, an injectable composition containing microparticles. The microparticles comprise a therapeutically effective amount of the active ingredient which is entrapped in a biodegradable or non-biodegradable polymer. Each possibility represents a separate embodiment of the invention. In various embodiments, the microparticles comprise dried water-in-oil-in-water (w/o/w) double emulsion droplets. The double emulsion droplets, according to the principles of the present invention, comprise an internal aqueous phase comprising a therapeutically effective amount of liraglutide or a pharmaceutically acceptable salt thereof; a water immiscible polymeric phase comprising a biodegradable carrier selected from the group consisting of polylactides, polyglycolides, polycaprolactones, and combinations thereof; and an external aqueous phase.

According to the principles of the present invention, liraglutide may be present in the composition in the form of free base or in the form of its salts or mixtures thereof. Each possibility represents a separate embodiment. Representative examples of salts include, but are not limited to, salts with suitable inorganic acids such as hydrochloric acid, hydrobromic acid, and the like. Each possibility represents a separate embodiment. Representative examples of salts also include, but are not limited to, salts with organic acids such as formic acid, acetic acid, propionic acid, lactic acid, tartaric acid, ascorbic acid, citric acid, and the like. Each possibility represents a separate embodiment. Representative examples of salts also include, but are not limited to, salts with bases such as triethanolamine, diethylamine, meglumine, arginine, alanine, leucine, diethylethanolamine, olamine, triethylamine, tromethamine, choline, trimethylamine, taurine, benzamine, methylamine, dimethylamine, trimethylamine, methylethanolamine, propylamine, isopropylamine, adenine, guanine, cytosine, thymine, uracil, thymine, xanthine, hypoxanthine, and the like. Each possibility represents a separate embodiment. According to further embodiments, pharmaceutically acceptable salts include acid addition salts such as those containing sulfate, phosphate, sulfamate, methanesulfonate, ethanesulfonate, benzenesulfonate, p-toluenesulfonate, cyclohexylsulfamate, and quinate. Each possibility represents a separate embodiment.

Salts according to the principles of the present invention may be prepared by, for example, reacting the free acid or free base forms with one or more equivalents of the appropriate base or acid, respectively, in a solvent or medium in which the salt is insoluble, or in a solvent such as water which is then removed in vacuo, by freeze-drying, or by exchanging the ions of an existing salt for another ion on a suitable ion exchange resin. Each possibility represents a separate embodiment of the invention.

In a currently preferred embodiment, liraglutide is present in the composition as the free base, i.e., liraglutide is not in the form of a salt. In another embodiment, liraglutide is present as an acetate salt.

According to various aspects and embodiments, liraglutide is present in the parenteral compositions disclosed herein in a therapeutically effective amount. As used herein, the term “therapeutically effective amount” is intended to qualify the amount of liraglutide, that provides the following responses after administration: stimulation of glucose-dependent insulin release and/or suppression of postprandial glucagon secretion in patients with type-2 diabetes. In some embodiments, liraglutide is present in the parenteral compositions disclosed herein at a dose of about 5 mg to about 100 mg, including each value within the specified range. Typical doses within the scope of the present invention include, but are not limited to, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. Each possibility represents a separate embodiment.

In certain embodiments, the dosage forms include, but are not limited to, biodegradable injectable depot systems such as, PLGA based injectable depot systems, non-PLGA based injectable depot systems, and injectable biodegradable gels or dispersions. Each possibility represents a separate embodiment of the invention. The term “biodegradable” as used herein refers to a component which erodes or degrades at its surfaces over time due, at least in part, to contact with substances found in the surrounding tissue fluids, or by cellular action. Suitable biodegradable or non-biodegradable depot systems within the scope of the present invention include, but are not limited to, systems comprising at least one of the following polymers: polyanhydrides; poly(sebacic acid) SA; poly(ricinoleic acid) RA; poly(fumaric acid), FA; poly(fatty acid dimmer), FAD; poly(terephthalic acid), TA; poly(isophthalic acid), IPA; poly(p-{carboxyphenoxy}methane), CPM; poly(p-{carboxyphenoxy} propane), CPP; poly(p-{carboxyphenoxy}hexane) CPH; polyamines, polyurethanes, polyesteramides, polyorthoesters {CHDM: cis/trans-cyclohexyl dimethanol, HD:1,6-hexanediol, DETOU: (3,9-diethylidene-2,4,8,10-tetraoxaspiro undecane)}; polydioxanones; polyhydroxybutyrates; polyalkylene oxalates; polyamides; polyesteramides; polyacetals; polyketals; polycarbonates; polyorthocarbonates; polysiloxanes; polyphosphazenes; succinates; hyaluronic acid; poly(malic acid); poly(amino acids); polyhydroxyvalerates; polyalkylene succinates; polyvinylpyrrolidone; polystyrene; synthetic cellulose esters; polyacrylic acids; polybutyric acid; triblock copolymers (PLGA-PEG-PLGA), triblock copolymers (PEG-PLGA-PEG), poly (N-isopropylacrylamide) (PNIPAAm), poly (ethylene oxide)-poly (propylene oxide)-poly (ethylene oxide) tri-block copolymers (PEO-PPO-PEO), poly valeric acid; polyethylene glycol; polyhydroxyalkylcellulose; chitin; chitosan; polyorthoesters and copolymers, terpolymers; lipids such as cholesterol, lecithin; poly(glutamic acid-co-ethyl glutamate) and the like, or mixtures thereof. Each possibility represents a separate embodiment of the invention.

Additional depot systems within the scope of the present invention include, but are not limited to, systems comprising at least one of the following polymers: poly (D,L-lactide-co-glycolide) (PLGA), poly (D,L-lactide) (PLA), polyglycolide (PGA), polycaprolactone (PCL), polyhydroxybutyrate, polyorthoesters, polyalkaneanhydrides, gelatin, collagen, oxidized cellulose, polyphosphazene, and any combination thereof. Each possibility represents a separate embodiment of the invention.

In some aspects and embodiments, the therapeutically effective amount of liraglutide or a pharmaceutically acceptable salt thereof is encapsulated in an oily phase comprising a biodegradable component which is a polymer such as, but not limited to, a polylactide, a polyglycolide, a polycaprolactone, and a mixture or combination thereof. Each possibility represents a separate embodiment. In particular, the biodegradable polymer comprises, but is not limited to, lactic acid-based polymers such as polylactides e.g. poly (D,L-lactide) i.e. PLA; glycolic acid-based polymers such as polyglycolide (PGA) e.g. Lactel® from Durect; poly (D,L-lactide-co-glycolide) i.e. PLGA, (Resomer® RG-504, Resomer® RG-502, Resomer® RG-504H, Resomer® RG-502H, Resomer® RG-504S, Resomer® RG-502S, from Boehringer, Lactel® from Durect); and polycaprolactones such as poly(ε-caprolactone) i.e. PCL (Lactel® from Durect). Each possibility represents a separate embodiment.

A currently preferred biodegradable polymer is a lactic acid-based polymer, more preferably polylactide, or poly (D, L-lactide-co-glycolide) i.e. PLGA. Another currently preferred biodegradable polymer is polycaprolactone (PCL). Yet another currently preferred biodegradable polymer is polylactic acid (PLA). Further currently preferred biodegradable polymer is PLA-PCL. An additional currently preferred biodegradable polymer is a mixture of PLGA with PLA-PCL. In one embodiment, the weight % ratio of PLGA to PLA-PCL is in the range of 9:1 to 1:9, including all iterations of ratios within the specified range. In another embodiment, the weight % ratio of PLGA to PLA-PCL is in the range of 9:1 to 7:3, including all iterations of ratios within the specified range. In yet another embodiment, the weight % ratio of PLGA to PLA-PCL is 8:2. Typically, the biodegradable polymer is present in an amount between about 10% and about 98% w/w of the solid composition (e.g. microparticles) including each value within the specified range. However, it is understood that the amount of biodegradable polymer is determined by parameters such as the duration of use and the like. In some embodiments, the lactic acid-based polymer has a monomer ratio of lactic acid to glycolic acid in the range of 100:0 to about 0:100, preferably 100:0 to about 10:90, including each value within the specified range. In one embodiment, the lactic acid-based polymer has a monomer ratio of lactic acid to glycolic acid of 80:20. In another embodiment, the lactic acid-based polymer has a monomer ratio of lactic acid to glycolic acid of 75:25. In yet another embodiment, the lactic acid-based polymer has a monomer ratio of lactic acid to glycolic acid of 50:50. In further embodiments, the biodegradable polymer has an average molecular weight of from about 1,000 to about 200,000 Daltons, including each value within the specified range.

Without being bound by a particular theory it is believed that the release of liraglutide from the depot formulation can occur by two different mechanisms. The first mechanism includes the release by diffusion through aqueous filled channels generated in the polymer matrix, such as by the dissolution of the biologically active agent, or by voids created by the removal of the polymer solvent during the preparation of the sustained release composition. Additional channels may be formed using a pore-former e.g. zinc oxide. The second mechanism includes the release of the biologically active agent due to degradation of the polymer. The rate of degradation can be controlled by tailoring polymer properties that influence its rate of hydration. These properties include, for instance, the ratio of lactide to glycolide comprising a polymer, the use of the L-isomer of a monomer instead of a racemic mixture, and the molecular weight of the polymer. These properties can affect hydrophilicity and crystallinity, which control the rate of hydration of the polymer. By altering the properties of the polymer, the contributions of diffusion and/or polymer degradation to biologically active agent release can be controlled. For example, increasing the glycolide content of a poly(lactide-co-glycolide) polymer and decreasing the molecular weight of the polymer can enhance the hydrolysis of the polymer and thus, provide an increased biologically active agent release from polymer erosion.

According to further aspects and embodiments, the emulsion droplets comprise an external aqueous phase. In currently preferred embodiments, the external aqueous phase further comprises a surfactant. Suitable surfactants include, but are not limited to, polyvinyl alcohol (PVA), polysorbates, polyethylene oxide-polypropylene oxide block copolymers, cellulose esters, and the like. Each possibility represents a separate embodiment. A currently preferred surfactant is polyvinyl alcohol (PVA). The external aqueous phase may further comprise a tonicity modifier, for example sodium chloride.

According to the principles of the present invention, the water-in oil-in water (w/o/w) double emulsion droplets are subsequently dried to provide dried microparticles. The dried microparticles can be administered as is. According to some aspects and embodiments, the dried microparticles are suspended in an inert oil, suitably a vegetable oil such as sesame, peanut, olive oil, or other acceptable carrier. Each possibility represents a separate embodiment. Preferably, the dried microparticles are suspended in an aqueous carrier, for example, an isotonic buffer solution at a pH of about 3.0 to about 7.0, more preferably of about 4.0 to about 6.0, and most preferably of about 4.0 to about 5.0, including each value within the specified ranges. These compositions may be sterilized by conventional sterilization techniques, or may be sterile filtered. The compositions may contain pharmaceutically acceptable auxiliary substances as required to approximate physiological conditions, such as pH buffering agents. Suitable buffers include, but are not limited to, sodium acetate/acetic acid buffers. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Each possibility represents a separate embodiment of the invention. Sodium chloride is preferred particularly for buffers containing sodium ions.

According to some embodiments, carriers or excipients can also be used to facilitate administration of the dosages of the present invention. Examples of carriers and excipients include, but are not limited to, calcium carbonate, calcium phosphate, various sugars such as lactose, or various types of starch, cellulose derivatives, gelatin, vegetable oils, polyethylene glycols and physiologically compatible solvents. Each possibility represents a separate embodiment of the invention.

According to various embodiments, solutions of the dosage forms may be thickened with a thickening agent such as, but not limited to, methylcellulose. They may be prepared in emulsified form, either water-in-oil or oil-in-water. Any of a wide variety of pharmaceutically acceptable emulsifying agents may be employed including, for example, acacia powder, a non-ionic surfactant (such as a Tween), or an ionic surfactant (such as alkali polyether alcohol sulfates or sulfonates, e.g., a Triton). Each possibility represents a separate embodiment of the invention.

According to additional embodiments, the pharmaceutically acceptable carrier is a liquid. According to further embodiments, the liquid is selected from the group consisting of an aqueous solvent or a non-aqueous solvent, emulsions and suspensions. Each possibility represents a separate embodiment. According to other embodiments, the liquid is an aqueous solvent selected from the group consisting of saline, dextrose solutions, and glycerol solutions. Each possibility represents a separate embodiment of the invention.

The compositions of the present invention may further comprise one or more pharmaceutically acceptable excipient(s) selected from, but not limited to, co-surfactants/solubilizers, solvents/co-solvents, water immiscible solvents, water, water miscible solvents, oily components, hydrophilic solvents, emulsifiers, preservatives, antioxidants, anti-foaming agents, stabilizers, buffering or pH adjusting agents, osmotic agents, pore forming agents, osmotic adjustment agents, or any other excipient known in the art. Each possibility represents a separate embodiment. Suitable co-surfactants or solubilizers include, but are not limited to, polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil, and the like or mixtures thereof. Each possibility represents a separate embodiment of the invention. Suitable solvents/co-solvents include, but not limited to, alcohols, triacetin, dimethyl isosorbide, glycofurol, propylene carbonate, water, dimethyl acetamide, and the like or mixtures thereof. Each possibility represents a separate embodiment of the invention. Suitable anti-foaming agents include, but are not limited to, silicon emulsions or sorbitan sesquioleate. Each possibility represents a separate embodiment of the invention. Suitable stabilizers to prevent or reduce the deterioration of the components in the compositions of the present invention include, but are not limited to, antioxidants such as glycine, α-tocopherol or ascorbate, BHA, BHT, and the like or mixtures thereof. Each possibility represents a separate embodiment of the invention. Suitable tonicity modifiers include, but are not limited to, mannitol, sodium chloride, and glucose. Each possibility represents a separate embodiment of the invention. Suitable buffering agents include, but are not limited to, acetates, phosphates, and citrates with suitable cations. Each possibility represents a separate embodiment of the invention.

According to certain embodiments, the parenteral pharmaceutical composition of the invention comprises at least one divalent metal ion. Suitable divalent metal ions within the scope of the present invention include, but are not limited to, zinc ions, calcium ions, magnesium ions, ferrous ions, and the like. Each possibility represents a separate embodiment of the invention. In some embodiments, the parenteral pharmaceutical composition of the invention comprises zinc ions in the form of zinc oxide.

The sustained release depot systems of the present invention can be prepared by any manner known in the art. Currently preferred is the incorporation of liraglutide or a pharmaceutically acceptable salt thereof into a colloidal delivery system, e.g., biodegradable microparticles, thus allowing release retardation by diffusion through polymeric walls of the particle and by polymer degradation in water media or biological fluids in the body.

According to some embodiments, the sustained-release microparticles of the present invention can be prepared in the form of injectable dried microparticles by a process known as the “double emulsification”. Briefly, a concentrated aqueous solution or suspension of liraglutide or a pharmaceutically acceptable salt thereof optionally comprising a surfactant (e.g. polyvinyl alcohol-PVA, polysorbates, polyethylene oxide-polypropylene oxide block copolymers, cellulose esters and the like) is prepared. The aqueous solution or suspension is then dispersed in a solution of a biodegradable or non-biodegradable polymer in a water-immiscible volatile organic solvent (e.g. methylene chloride, chloroform and the like). The thus obtained “water-in-oil” (w/o) emulsion is then dispersed in a continuous external water phase containing a surfactant (e.g. polyvinyl alcohol-PVA, polysorbates, polyethylene oxide-polypropylene oxide block copolymers, cellulose esters and the like) to form “water-in oil-in water (w/o/w) double emulsion” droplets. After evaporation of the organic solvent, the microparticles solidify and are collected by filtration or centrifugation. The terms “oil phase” and “water-immiscible phase” may be used interchangeably herein. In some embodiments, the weight % ratio of the internal aqueous phase to the water-immiscible phase ranges from about 1:4 to about 1:10, including all iterations of ratios within the specified range. In other embodiments, the weight % ratio of the water-immiscible phase to the external aqueous phase ranges from about 1:5 to about 1:17, including all iterations of ratios within the specified range. In one currently preferred embodiment, the weight % ratio of the internal aqueous phase to the water-immiscible phase to the external aqueous phase is about 1:4.5:45. In another currently preferred embodiment, the weight % ratio of the internal aqueous phase to the water-immiscible phase to the external aqueous phase is about 1:9:90. In yet another currently preferred embodiment, the weight % ratio of the internal aqueous phase to the water-immiscible phase to the external aqueous phase is about 1:8:80. In an additional currently preferred embodiment, the weight % ratio of the internal aqueous phase to the water-immiscible phase to the external aqueous phase is about 1:6:90.

The collected microparticles (MPs) are washed with purified water to eliminate most of the surfactant and free peptide and centrifuged again. The washed MPs are collected and lyophilized without additives or with the addition of a cryoprotectant (mannitol) to facilitate their subsequent reconstitution.

According to further embodiments, the particle size of the “water-in oil-in water (w/o/w) double emulsion” can be determined by various parameters including, but not limited to, the amount of applied force at this step, the speed of mixing, surfactant type and concentration, etc. Suitable particle sizes range from about 1 to about 100 μm, including each value within the specified range.

### Methods of Use

The present invention provides a method for treating or delaying the progression or onset of diabetes, especially type-2 diabetes, including complications of diabetes, such as retinopathy, neuropathy, nephropathy and delayed wound healing, and related diseases such as insulin resistance (impaired glucose homeostasis), hyperglycemia, hyperinsulinemia, elevated blood levels of fatty acids or glycerol, obesity, hyperlipidemia including hypertriglyceridemia, Syndrome X, atherosclerosis, hypertension, and cardiovascular events such as cardiovascular death, non-fatal myocardial infarction, or non-fatal stroke, and for increasing high density lipoprotein levels. The method comprises administering a long-acting parenteral pharmaceutical composition comprising liraglutide or a pharmaceutically acceptable salt thereof and a biodegradable carrier selected from the group consisting of polylactides, polyglycolides, polycaprolactones, and combinations thereof, wherein the composition is in depot form suitable for implantation at a medically acceptable location in a subject in need thereof, wherein the composition achieves average human steady-state plasma concentrations (C_{ss,avg}) of liraglutide of at least about 5 nmol/L for about 1 week to about 28 days after a single administration. In some embodiments, the present invention relates to a sustained release pharmaceutical composition comprising a therapeutically effective amount of liraglutide or any other pharmaceutically acceptable salt thereof, for use in treating type-2 diabetes mellitus.

In other embodiments, the present invention provides a method of treating type-2 diabetes mellitus comprising the step of administering to a subject in need thereof a long-acting parenteral pharmaceutical composition comprising liraglutide or a pharmaceutically acceptable salt thereof, a biodegradable carrier selected from the group consisting of polylactides, polyglycolides, polycaprolactones, and combinations thereof, and at least one divalent metal ion, wherein the composition is in depot form suitable for implantation at a medically acceptable location in a subject in need thereof, wherein the composition achieves average human steady-state plasma concentrations (C_{ss,avg}) of liraglutide of at least about 5 nmol/L for about 1 week to about 28 days after a single administration to a subject. In one embodiment, the composition achieves average human steady-state plasma concentrations (C_{ss,avg}) of liraglutide of between about 5 nmol/L and about 45 nmol/L for about 1 week to about 28 days after a single administration to a subject. In certain embodiments, the average human steady-state plasma concentrations (C_{ss,avg}) of liraglutide of between about 5 nmol/L and about 45 nmol/L are achieved 2 days or more after a single parenteral administration.

In further embodiments, the present invention provides a method of treating type-2 diabetes mellitus, comprising the step of administering to a subject in need thereof a long-acting parenteral pharmaceutical composition comprising microparticles comprising dried water-in-oil-in-water (w/o/w) double emulsion droplets comprising an internal aqueous phase comprising a therapeutically effective amount of liraglutide or a pharmaceutically acceptable salt thereof; a water immiscible polymeric phase comprising a biodegradable carrier selected from the group consisting of polylactides, polyglycolides, polycaprolactones, and combinations thereof; and an external aqueous phase, wherein the composition is in depot form suitable for implantation at a medically acceptable location in a subject in need thereof at a frequency of once weekly to once every six months and provides a twenty-four hour liraglutide burst release of less than 15% of the administered dose following administration. The term “treating” as used herein with reference to type-2 diabetes refers to suppression or alleviation of short- and long-term symptoms and complications associated with type-2 diabetes, for example hyperglycemia, and any one of the aforementioned complications. In various embodiments, the compositions disclosed herein reduce fasting glucose levels by at least about 20%, preferably by at least about 30%. In other embodiments, the compositions disclosed herein reduce fed glucose levels by at least about 10%, preferably by at least about 15%, and more preferably by at least about 20%. In further embodiments, the compositions disclosed herein reduce hemoglobin A1C (HbA1C) levels in said subject by at least about 20%, preferably by at least about 30%.

In some embodiments, the present invention relates to a method of reducing fasting glucose levels in a subject by at least about 20% for a time period between about 1 week and about 28 days after a single administration, the method comprising the step of administering to a subject in need thereof a long-acting parenteral pharmaceutical composition comprising liraglutide or a pharmaceutically acceptable salt thereof and a biodegradable carrier selected from the group consisting of polylactides, polyglycolides, polycaprolactones, and combinations thereof, wherein the composition is in depot form suitable for implantation at a medically acceptable location in a subject in need thereof. In certain embodiments, the present invention relates to a sustained release pharmaceutical composition comprising a therapeutically effective amount of liraglutide or a pharmaceutically acceptable salt thereof, for use in reducing fasting glucose levels. In various embodiments, the fasting glucose levels are reduced by at least 21%, by at least 22%, by at least 23%, by at least 24%, by at least 25%, by at least 26%, by at least 27%, by at least 28%, by at least 29%, or by at least 30% for a period of at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, or even longer after a single administration. Each possibility represents a separate embodiment. In certain embodiments, the fasting glucose levels are reduced by at least 30% up to 14 days after administration of the composition disclosed herein. Within the scope of the present invention are fasting glucose levels after a single parenteral administration of the composition of the invention of between about 70 and about 400 mg/dL, including each value within the specified range. For example, fasting glucose levels after a single parenteral administration include, but are not limited to, about 70 mg/dL, about 75 mg/dL, about 80 mg/dL, about 85 mg/dL, about 90 mg/dL, about 95 mg/dL, about 100 mg/dL, about 110 mg/dL, about 120 mg/dL, about 130 mg/dL, about 140 mg/dL, about 150 mg/dL, about 160 mg/dL, about 170 mg/dL, about 180 mg/dL, about 190 mg/dL, about 200 mg/dL, about 210 mg/dL, about 220 mg/dL, about 230 mg/dL, about 240 mg/dL, about 250 mg/dL, about 260 mg/dL, about 270 mg/dL, about 280 mg/dL, about 290 mg/dL, about 300 mg/dL, about 310 mg/dL, about 320 mg/dL, about 330 mg/dL, about 340 mg/dL, about 350 mg/dL, about 360 mg/dL, about 370 mg/dL, about 380 mg/dL, about 390 mg/dL, and about 400 mg/dL. Each possibility represents a separate embodiment.

In other embodiments, the present invention relates to a method of reducing fed glucose levels in a subject by at least about 10% for a time period between about 1 week and about 28 days after a single administration, the method comprising the step of administering to a subject in need thereof a long-acting parenteral pharmaceutical composition comprising liraglutide or a pharmaceutically acceptable salt thereof and a biodegradable carrier selected from the group consisting of polylactides, polyglycolides, polycaprolactones, and combinations thereof, wherein the composition is in depot form suitable for implantation at a medically acceptable location in a subject in need thereof. In certain embodiments, the present invention relates to a sustained release pharmaceutical composition comprising a therapeutically effective amount of liraglutide or a pharmaceutically acceptable salt thereof, for use in reducing fed glucose levels. In some embodiments, the fed glucose levels are reduced by at least 10%, by at least 11%, by at least 12%, by at least 13%, by at least 14%, by at least 15%, by at least 16%, by at least 17%, by at least 18%, by at least 19%, or by at least 20% for a period of at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, or even longer after a single administration. Each possibility represents a separate embodiment. In certain embodiments, the fed glucose levels are reduced by at least 20% up to 10 days after administration of the compositions disclosed herein. Within the scope of the present invention are fed glucose levels after a single parenteral administration of the composition of the present invention of between about 120 and about 650 mg/dL, including each value within the specified range. For example, fed glucose levels after a single parenteral administration include, but are not limited to about 120 mg/dL, about 130 mg/dL, about 140 mg/dL, about 150 mg/dL, about 160 mg/dL, about 170 mg/dL, about 180 mg/dL, about 190 mg/dL, about 200 mg/dL, about 210 mg/dL, about 220 mg/dL, about 230 mg/dL, about 240 mg/dL, about 250 mg/dL, about 260 mg/dL, about 270 mg/dL, about 280 mg/dL, about 290 mg/dL, about 300 mg/dL, about 310 mg/dL, about 320 mg/dL, about 330 mg/dL, about 340 mg/dL, about 350 mg/dL, about 360 mg/dL, about 370 mg/dL, about 380 mg/dL, about 390 mg/dL, about 400 mg/dL, about 410 mg/dL, about 420 mg/dL, about 430 mg/dL, about 440 mg/dL, about 450 mg/dL, about 460 mg/dL, about 470 mg/dL, about 480 mg/dL, about 490 mg/dL, about 500 mg/dL, about 510 mg/dL, about 520 mg/dL, about 530 mg/dL, about 540 mg/dL, about 550 mg/dL, about 560 mg/dL, about 570 mg/dL, about 580 mg/dL, about 590 mg/dL, about 600 mg/dL, about 610 mg/dL, about 620 mg/dL, about 630 mg/dL, about 640 mg/dL, and about 650 mg/dL. Each possibility represents a separate embodiment.

In another embodiment, the present invention relates to a method of reducing hemoglobin A1C (HbA1C) levels in a subject by at least about 20% for a time period between about 1 week and about 28 days after a single administration, the method comprising the step of administering to a subject in need thereof a long-acting parenteral pharmaceutical composition comprising liraglutide or a pharmaceutically acceptable salt thereof and a biodegradable carrier selected from the group consisting of polylactides, polyglycolides, polycaprolactones, and combinations thereof, wherein the composition is in depot form suitable for implantation at a medically acceptable location in a subject in need thereof. In some embodiments, the present invention relates to a sustained release pharmaceutical composition comprising a therapeutically effective amount of liraglutide or a pharmaceutically acceptable salt thereof, for use in reducing HbA1C levels. In certain embodiments, the HbA1C levels are reduced by at least 21%, by at least 22%, by at least 23%, by at least 24%, by at least 25%, by at least 26%, by at least 27%, by at least 28%, by at least 29% or by at least 30% for a period of at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, or even longer after a single administration. Each possibility represents a separate embodiment. Within the scope of the present invention are hemoglobin A1C (HbA1C) levels after a single parenteral administration of the composition of the invention of between about 4% and about 10.5%, including each value within the specified range. For example, hemoglobin A1C (HbA1C) levels after a single parenteral administration include, but are not limited to about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, about 10.0%, about 10.1%, about 10.2%, about 10.3%, about 10.4%, and about 10.5%. Each possibility represents a separate embodiment.

In addition, the present invention provides a method of treating Parkinson's Disease by parenteral administration of a composition comprising liraglutide or a pharmaceutically acceptable salt thereof and a biodegradable carrier selected from the group consisting of polylactides, polyglycolides, polycaprolactones, and combinations thereof, wherein the composition is in depot form suitable for implantation at a medically acceptable location in a subject in need thereof, wherein the composition achieves average human steady-state plasma concentrations (C_{ss,avg}) of liraglutide of at least about 5 nmol/L for about 1 week to about 28 days after a single administration. In some embodiments, the present invention relates to a sustained release pharmaceutical composition comprising a therapeutically effective amount of liraglutide or a pharmaceutically acceptable salt thereof, for use in the treatment of Parkinson's Disease.

In various embodiments, the present invention provides a method of treating Parkinson's Disease, comprising the step of administering to a subject in need thereof a long-acting parenteral pharmaceutical composition comprising liraglutide or a pharmaceutically acceptable salt thereof, a biodegradable carrier selected from the group consisting of polylactides, polyglycolides, polycaprolactones, and combinations thereof, and at least one divalent metal ion, wherein the composition is in depot form suitable for implantation at a medically acceptable location in a subject in need thereof, wherein the composition achieves average human steady-state plasma concentrations (C_{ss,avg}) of liraglutide of at least about 5 nmol/L for about 1 week to about 28 days after a single administration to a subject. In one embodiment, the composition achieves average human steady-state plasma concentrations (C_{ss,avg}) of liraglutide of between about 5 nmol/L and about 45 nmol/L for about 1 week to about 28 days after a single administration to a subject.

In further embodiments, the present invention provides a method of treating Parkinson's Disease, comprising the step of administering to a subject in need thereof a long-acting parenteral pharmaceutical composition comprising microparticles comprising dried water-in-oil-in-water (w/o/w) double emulsion droplets comprising an internal aqueous phase comprising a therapeutically effective amount of liraglutide or a pharmaceutically acceptable salt thereof; a water immiscible polymeric phase comprising a biodegradable carrier selected from the group consisting of polylactides, polyglycolides, polycaprolactones, and combinations thereof; and an external aqueous phase, wherein the composition is in depot form suitable for implantation at a medically acceptable location in a subject in need thereof at a frequency of once weekly to once every six months and provides a twenty-four hour liraglutide burst release of less than 15% of the administered dose following administration.

As used herein, the term “treating” with reference to Parkinson's Disease refers to reversing, alleviating, ameliorating, inhibiting, slowing down and/or stopping the progression or severity of at least one adverse effect or symptom of Parkinson's Disease including, for example, those associated with impaired motoric function.

It is understood that the amount of the liraglutide administered will be determined by a physician, according to various parameters including the chosen route of administration, the age, weight, and the severity of the patient's disease and symptoms. The required plasma concentrations of liraglutide that provide therapeutic efficacy can be determined, for example, from in-vitro and in-vivo models as is known in the art. According to some specific exemplary embodiments, the average plasma concentration of liraglutide is generally between about 0.1 μg/ml and about 100 μg/ml, including each value within the specified range. According to further embodiments, the average plasma concentration of liraglutide is generally between about 0.1 μg/ml and about 50 μg/ml, including each value within the specified range. According to further embodiments, the average plasma concentration of liraglutide is generally between about 0.3 μg/ml and about 40 μg/ml, including each value within the specified range. According to further embodiments, the average plasma concentration of liraglutide is generally between about 1 μg/ml and about 35 μg/ml, including each value within the specified range.

According to further embodiments, the average plasma concentration of liraglutide is generally between about 1 ng/ml and about 1,000 ng/ml, including each value within the specified range. According to other embodiments, the average plasma concentration of liraglutide is generally between about 1 ng/ml and about 500 ng/ml, including each value within the specified range. According to yet other embodiments, the average plasma concentration of liraglutide is generally between about 1 ng/ml and about 250 ng/ml, including each value within the specified range. According to additional embodiments, the average plasma concentration of liraglutide is generally between about 50 ng/ml and about 500 ng/ml, including each value within the specified range. According to particular embodiments, the average plasma concentration of liraglutide is generally between about 100 ng/ml and about 500 ng/ml, including each value within the specified range. According to some embodiments, the average plasma concentration of liraglutide is generally between about 100 ng/ml and about 250 ng/ml, including each value within the specified range.

The compositions of the invention achieve average human steady-state plasma concentrations (C_{ss,avg}) of liraglutide of at least about 5 nmol/L for about 1 week to about 28 days after a single administration. In some embodiments, the compositions of the invention achieve average human steady-state plasma concentrations (C_{ss,avg}) of liraglutide of between about 5 nmol/L and about 45 nmol/L for about 1 week to about 28 days after a single administration. In other embodiments, the compositions of the invention achieve average human steady-state plasma concentrations (C_{ss,avg}) of liraglutide of between about nmol/L and about 40 nmol/L for about 1 week to about 28 days after a single administration. In yet other embodiments, the compositions of the invention achieve average human steady-state plasma concentrations (C_{ss,avg}) of liraglutide of between about nmol/L and about 35 nmol/L for about 1 week to about 28 days after a single administration. In further embodiments, the compositions of the invention achieve average human steady-state plasma concentrations (C_{ss,avg}) of liraglutide of between about 5 nmol/L and about 30 nmol/L for about 1 week to about 28 days after a single administration. In additional embodiments, the compositions of the invention achieve average human steady-state plasma concentrations (C_{ss,avg}) of liraglutide of between about nmol/L and about 40 nmol/L for about 1 week to about 28 days after a single administration. In some embodiments, the compositions of the invention achieve average human steady-state plasma concentrations (C_{ss,avg}) of liraglutide of between about 15 nmol/L and about 40 nmol/L for about 1 week to about 28 days after a single administration. In particular embodiments, the compositions of the invention achieve average human steady-state plasma concentrations (C_{ss,avg}) of liraglutide of between about nmol/L and about 20 nmol/L for about 1 week to about 28 days after a single administration. In some embodiments, the compositions of the invention achieve average human steady-state plasma concentrations (C_{ss,avg}) of liraglutide of between about 5 nmol/L and about 10 nmol/L for about 1 week to about 28 days after a single administration. In some embodiments, the compositions of the invention achieve average human steady-state plasma concentrations (C_{ss,avg}) of liraglutide of between about 10 nmol/L and about 20 nmol/L for about 1 week to about 28 days after a single administration. In some embodiments, the compositions of the invention achieve average human steady-state plasma concentrations (C_{ss,avg}) of liraglutide of between about 5 nmol/L and about 45 nmol/L for a time period starting two days (i.e. 48 hours) after a single administration up to about 28 days after said single administration.

The compositions of the invention provide a twenty-four hour liraglutide burst release of less than 15% of the administered dose following administration. In some embodiments, the compositions of the invention provide a twenty-four hour liraglutide burst release of less than 14% of the administered dose following administration. In other embodiments, the compositions of the invention provide a twenty-four hour liraglutide burst release of less than 13% of the administered dose following administration. In yet other embodiments, the compositions of the invention provide a twenty-four hour liraglutide burst release of less than 12% of the administered dose following administration. In additional embodiments, the compositions of the invention provide a twenty-four hour liraglutide burst release of less than 11% of the administered dose following administration. In further embodiments, the compositions of the invention provide a twenty-four hour liraglutide burst release of less than 10% of the administered dose following administration. In particular embodiments, the compositions of the invention provide a twenty-four hour liraglutide burst release of less than 9% of the administered dose following administration. In specific embodiments, the compositions of the invention provide a twenty-four hour liraglutide burst release of less than 8% of the administered dose following administration. In various embodiments, the compositions of the invention provide a twenty-four hour liraglutide burst release of less than 7% of the administered dose following administration. In certain embodiments, the compositions of the invention provide a twenty-four hour liraglutide burst release of less than 6% of the administered dose following administration. In exemplary embodiments, the compositions of the invention provide a twenty-four hour liraglutide burst release of less than 5% of the administered dose following administration.

According to further embodiments, the compositions of the present invention provide equal or superior therapeutic efficacy to the commercially available daily or weekly injectable dosage forms of the liraglutide, with reduced incidence of side effects and/or with reduced severity of side effects. Each possibility represents a separate embodiment of the invention.

According to some embodiments, the parenteral pharmaceutical depot composition of this invention can be administered in vivo to a subject in need thereof. In some embodiments, the “subject” to which the depot composition is administered is a mammal, preferably, but not limited to, a human.

As used herein and in the appended claims, the term “about” refers to ±10%.

As used herein and in the appended claims, the singular forms “a”, “an”, and “the” include plural references unless the context clearly dictates otherwise. Thus, for example, reference to “a biodegradable carrier” includes a plurality of such carriers. It should be noted that the term “and” or the term “or” are generally employed in its sense including “and/or” unless the context clearly dictates otherwise.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1: PLGA Injectable Liraglutide Microparticles

Liraglutide microspheres were prepared using water/oil/water (w1/o/w2) double emulsion solvent evaporation method.

### Preparation Proceedings

1. Aqueous phase (w1) (60 mg liraglutide powder dissolved in 300 μl water), was emulsified with 3 ml solution of PLGA polymer in dichloromethane (10% w/w) by ultrasonication, using sonical power of 30 w 10/10 sec. under cooling for 2 min.
2. The primary water-in-oil (w1/o) emulsion was poured into 30 ml of w2, an aqueous solution containing 0.5% PVA. This resulted in a coarse double emulsion (w1/o/w2) formed by homogenization with a mixing speed of 3,000 rpm for 1.5 min. at room temperature (r.t.).
3. The double emulsion was stirred for 5-14 hr. at room temperature and the resulting microspheres were then collected by centrifugation for 5 min. at 1,500 rpm, washed with distilled water to remove excessive emulsifier and the purified microspheres were collected and resuspended. The final suspension was then freeze-dried to obtain a fine powder (0.02 mbar, −51° C. for 72 hr).

### Example 2: PLA-PCL/PLGA Injectable Liraglutide Microparticles

Polymer microspheres were prepared using water/oil/water (w1/o/w2) double emulsion solvent evaporation method similar to Example 1.

### Preparation Proceedings

1. Aqueous phase (w1) (30 mg liraglutide powder dissolved in 300 μl water 0.5% PVA), was emulsified with 3 ml solution of PLA-PCL/PLGA polymer (2:8 respectively) in dichloromethane (10% w/w) by ultrasonication, using sonical power of 30 w 10/10 sec. under cooling for 2 min.
2. The primary water-in-oil (w1/o) emulsion was poured into 30 ml of w2, an aqueous solution containing 0.5% PVA. This resulted in a coarse double emulsion (w1/o/w2), formed by homogenization at a speed of 3,000 rpm for 1 min. at room temperature.
3. The double emulsion was stirred for 5-14 hr. at room temperature and the resulting microspheres were then collected by centrifugation for 5 min. at 1,500 rpm, washed with distilled water to remove excessive emulsifier and the purified microspheres were collected and resuspended. The final suspension was then freeze-dried to obtain a fine powder (0.02 mbar, −51° C. for 72 hr).

### Example 3: PLA Injectable Liraglutide Microparticles

Polymer microspheres were prepared using water/oil/water (w1/o/w2) double emulsion solvent evaporation method similar to Example 1.

### Preparation Proceedings

1. Aqueous phase (w1) (30 mg liraglutide powder dissolved in 300 μl sodium acetate solution 0.1M, pH 8.2 0.5% PVA), was emulsified with 3 ml solution of PLA polymer in dichloromethane (10% w/w) by ultrasonication, using sonical power of 30 w 10/10 sec. under cooling for 2 min.
2. The primary water-in-oil (w1/o) emulsion was poured into 30 ml of w2, an aqueous solution containing 0.5% PVA and 0.75 g NaCl. This resulted in a coarse double emulsion (w1/o/w2), formed by using a homogenizer with a mixing speed of 3,000 rpm for 1.5 min. at room temperature.
3. The double emulsion was stirred for 5-14 hr. at room temperature and the resulting microspheres were then collected by centrifugation for 5 min. at 1,500 rpm, washed with distilled water to remove excessive emulsifier and the purified microspheres were collected and resuspended. The final suspension was then freeze-dried to obtain a fine powder (0.02 mbar, −51° C. for 72 hr).

### Example 4: PLA-PCL Injectable Liraglutide Microparticles

Polymer microspheres were prepared using water/oil/water (w1/o/w2) double emulsion solvent evaporation method similar to Example 3. The aqueous phase (w1) (30 mg liraglutide powder dissolved in 300 μl sodium acetate solution 0.1M, pH 8.2 0.5% PVA), was emulsified with 3 ml solution of PLA-PCL polymer in dichloromethane (10% w/w) by ultrasonication, using sonical power of 30 w 10/10 sec. under cooling for 2 min. The rest of the procedure was equivalent to Example 3.

### Example 5: PLGA 75:25 (L/G) Injectable Liraglutide Microparticles

Polymer microspheres were prepared using water/oil/water (w1/o/w2) double emulsion solvent evaporation method similar to Example 2. The aqueous phase (w1) (30 mg liraglutide powder dissolved in 300 μl sodium acetate solution 0.1M, pH 8.2), was emulsified with 2 ml solution of PLGA polymer 75:25 (L/G) in dichloromethane (10% w/w) by ultrasonication, using sonical power of 30 w 10/10 sec. under cooling for 2 min. The rest of the procedure was equivalent to Example 2.

### Example 6: PLGA 75:25 (L/G) Injectable Liraglutide Microparticles Containing a Pore-Former

Polymer microspheres were prepared using water/oil/water (w1/o/w2) double emulsion solvent evaporation method similar to Example 2. The aqueous phase (w1) (30 mg liraglutide powder dissolved in 300 μl sodium acetate solution 0.1M, pH 8.2), was emulsified with 2 ml solution of PLGA polymer 75:25 (L/G) in dichloromethane (10% w/w, with 15 mg of zinc oxide) by ultrasonication, using sonical power of 30 w 10/10 sec. under cooling for 2 min. The rest of the procedure was equivalent to Example 2.

### Example 7: PLGA Liraglutide Microparticles Prepared by Oil/Water Single Emulsion

Polymer microspheres were prepared using an oil/water (o/w) single emulsion solvent evaporation method.

### Preparation Proceedings

1. A solution of liraglutide in organic solvent (120 mg of liraglutide was added to 10 ml solution of DCM:MeOH 9:1) was prepared, 2.5 ml of the above liraglutide solution was added to 3 ml polymer solution 20% PLGA in dichloromethane.
2. The inner phase was sonicated for 30 min. to reach good solubility. The oily solution was poured into 30 ml of an aqueous solution containing 0.5% PVA. This resulted in a coarse single emulsion (o/w) via mixing at 3,000 rpm for 2 min.
3. The emulsion was stirred overnight at room temperature and the resultant microspheres were then collected by centrifugation for 15 min. at 1,000 rpm followed by washing and freeze-drying.

### Example 8: PLGA Liraglutide Microparticles Prepared by Oil/Water Single Emulsion in NMP:DCM

Polymer microspheres were prepared using an oil/water single emulsion solvent evaporation method similar to Example 7. A solution of liraglutide in organic solvent (50 mg of liraglutide was added to 2 ml solution of NMP:DCM 1:1) was prepared, 2 ml of the above liraglutide solution was added to 3 ml polymer solution, 10% PLGA in dichloromethane. The rest of the procedure was equivalent to Example 7.

### Example 9: PLGA Liraglutide Injectable Microparticles Containing a Solubilizing Agent

Polymer microspheres were prepared using water/oil/water (w1/o/w2) double emulsion solvent evaporation method similar to Example 3. The aqueous phase (w1) (30 mg liraglutide powder dissolved in 300 μl sodium acetate solution 0.1M, pH 8.2 and poloxamer 188 (3%)), was emulsified with 4.5 ml solution of PLGA polymer in dichloromethane (6% w/w) by ultrasonication, using sonical power of 30 w 10/10 sec. under cooling for 2 min. The w1/o emulsion was poured into 60 ml of w2, an aqueous solution containing 0.5% PVA and 1.5 g NaCl. This resulted in a coarse double emulsion (w1/o/w2) formed by homogenization with a mixing speed of 3,000 rpm for 1 min. at room temperature. The rest of the procedure was equivalent to Example 3.

### Example 10: PLGA In Situ Implant

A PLGA polymer solution in NMP (30% w/w) was heated for 6 hr. at 50° C. until dissolution. Liraglutide dry powder (65 mg) was added to the gel and the gel was heated for another half hour at 50° C. The solution is used as is for administration.

### Example 11: Low-Burst PLGA or PLGA/PLA-PCL Liraglutide Microparticles Prepared by w/o/w Double Emulsification

A. Low-Burst PLGA Liraglutide Microparticles

Liraglutide microspheres were prepared using water/oil/water (w1/o/w2) double emulsion solvent evaporation method.

### Preparation Proceedings

1. Aqueous phase (w1) (2 G liraglutide powder dissolved in 20 ml water), was emulsified with 100 G solution of PLGA polymer in dichloromethane (10% w/w) by homogenization with a mixing speed of 14,200 rpm for 2 min. at room temperature (r.t.).
2. The primary water-in-oil (w1/o) emulsion was poured into 1 L of w2, an aqueous solution containing 0.5% PVA and 0.8 or 3% NaCl. This resulted in a coarse double emulsion (w1/o/w2) formed by homogenization with a mixing speed of 3,000 rpm for 1 min, then 4,000 rpm for 4 more min. at room temperature (r.t.).
3. The double emulsion was stirred for 15 hr. at room temperature and the resulting microspheres were then collected by centrifugation for 5 min. at 1,500 rpm, washed with distilled water to remove excessive emulsifier and the purified microspheres were collected and resuspended. The final suspension was then freeze-dried to obtain a fine powder (0.02 mbar, −51° C. for 72 hr).

B. Low-Burst PLGA/PLA-PCL Liraglutide Microparticles

Liraglutide microspheres were prepared using water/oil/water (w1/o/w2) double emulsion solvent evaporation method.

### Preparation Proceedings

1. Aqueous phase (w1) (1 G liraglutide powder dissolved in 10 ml water), was emulsified with 100 G solution of PLA-PCL/PLGA polymer (2:8 respectively) in dichloromethane (10% w/w) by homogenization with a mixing speed of 14,200 rpm for 2 min. at room temperature (r.t.).
2. The primary water-in-oil (w1/o) emulsion was poured into 1 L of w2, an aqueous solution containing 0.5% PVA and 0.8 or 3% NaCl. This resulted in a coarse double emulsion (w1/o/w2) formed by homogenization with a mixing speed of 3,000 rpm for 1 min then 4,000 rpm for 4 more min. at room temperature (r.t.).
3. The double emulsion was stirred for 15 hr. at room temperature and the resulting microspheres were then collected by centrifugation for 5 min. at 1,500 rpm, washed with distilled water to remove excessive emulsifier and the purified microspheres were collected and resuspended. The final suspension was then freeze-dried to obtain a fine powder (0.02 mbar, −51° C. for 72 hr).

### Example 12: Comparison of Liraglutide Formulations in an Animal Model of Diabetes

The objective of this study was to test the pharmacokinetic and pharmacodynamic effect of various formulations of liraglutide in genetically diabetic male db/db mice.

Treatment Groups: 1. Naïve control normal mice (n=6)
2. Placebo (vehicle) control db/db mice (n=8)
3. Liraglutide API (L-API), 200 mg/kg, subcutaneous (SC), daily injections (n=10)
4. PLGA encapsulated liraglutide depot (LIR-110919), dosed at 156.3 mg/kg, intramuscular (IM) on Day-1 only (n=10)
5. PCL-PLA/PLGA encapsulated liraglutide depot (LIR-221019), dosed 266.7 mg/kg, intramuscular (IM) on Day-1 only (n=10)

### Formulation Preparation:

L-API: 20 mg of liraglutide was dissolved in 100 mL of water for injection.
PLGA-liraglutide depot: 187.6 mg of PLGA-liraglutide microparticles prepared according to Example 1 were dissolved in 1.2 mL of water for injection.
PCL-PLA/PLGA-liraglutide depot: 266.7 mg of PLA-PCL/PLGA liraglutide microparticles prepared according to Example 2 were dissolved in 1 mL of water for injection.

### Evaluated Parameters:

Ad lib fed blood glucose levels were recorded every 3^{rd} day throughout the study period i.e. on Day-1, Day-3, Day-6, Day-9, Day-12, Day-15, Day-18, Day-21, Day-24, and Day-27. Samples were collected 3 hr. post treatment.
On Day-14 and Day-28, blood glucose levels were monitored in overnight fasted animals (˜14-16 hr.).
Samples for the measurement of liraglutide-API levels by bioanalysis were collected on Day-1, Day-7, Day-14, Day-21, and Day-28 at 0 hr. (before treatment) and 3 hr. post treatment.
On Day-1 and Day-28, blood samples were collected for the measurement of HbA1C levels.
Body weight and feed intake were recorded every day throughout treatment period.

### Results:

Effect of Test Formulations on Fasting Blood Glucose in db/db Mice

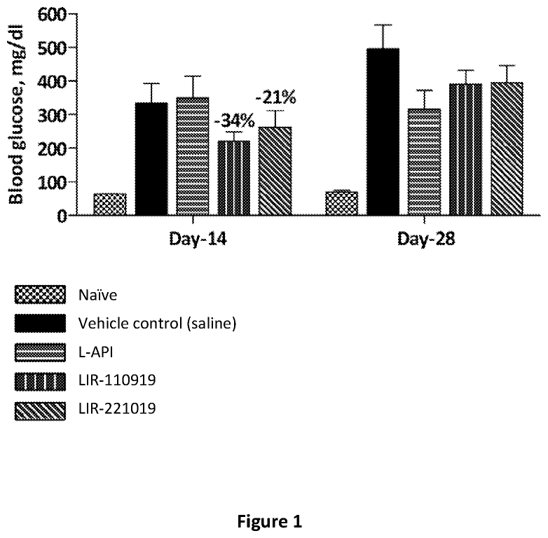

The effect of the test formulations on fasting blood glucose levels is shown in FIG. 1. db/db mice showed statistically significant higher levels of fasting blood glucose on Day-14 and Day-28 as compared to normal mice. L-API at 200 μg/kg, SC dose, administered daily did not show significant change in fasting blood glucose levels on Day-14 as compared to vehicle control. LIR-110919 (PLGA-liraglutide depot) at 156.3 mg/kg, IM dose administered on Day-1 only, showed a trend towards reduction in fasting blood glucose (−34.1%) on Day-14 as compared to vehicle control and −37% as compared to L-API. LIR-221019 (PCL-PLA/PLGA liraglutide depot) at 266.7 mg/kg, IM dose administered on Day-1 only, showed a trend towards reduction in fasting blood glucose (−21.1%) on Day-14 as compared to vehicle control and −25% as compared to L-API. The results are summarized in Table 1.

TABLE 1

| | Day 14 | Day 14 | Day 28 | Day 28 |
|---|---|---|---|---|
| | Mean ± SEM | % change vs. | mean ± SEM | % change vs. |
| Group | (mg/dL) | vehicle control | (mg/dL) | vehicle control |
| Control | 63.7 ± 1.4 | — | 70.2 ± 4.8 | — |
| (Naïve) | | | | |
| db/db vehicle | 333.5 ± 59.3 | — | 495.3 ± 72.0 | — |
| control | | | | |
| L-API | 349.0 ± 65.2 | +4.7% | 317.4 ± 54.7 | −35.9% |
| LIR-110919 | 219.9 ± 29.3 | −34.1% | 390.9 ± 41.0 | −21.1% |
| (PLGA- | | | | |
| liraglutide | | | | |
| depot) | | | | |
| LIR-221019 | 263.2 ± 48.7 | −21.1% | 395.5 ± 50.4 | −20.1% |
| (PCL-PLA/ | | | | |
| PLGA | | | | |
| liraglutide | | | | |
| depot) | | | | |

Effect of Test Formulations on Fed Blood Glucose in db/db Mice

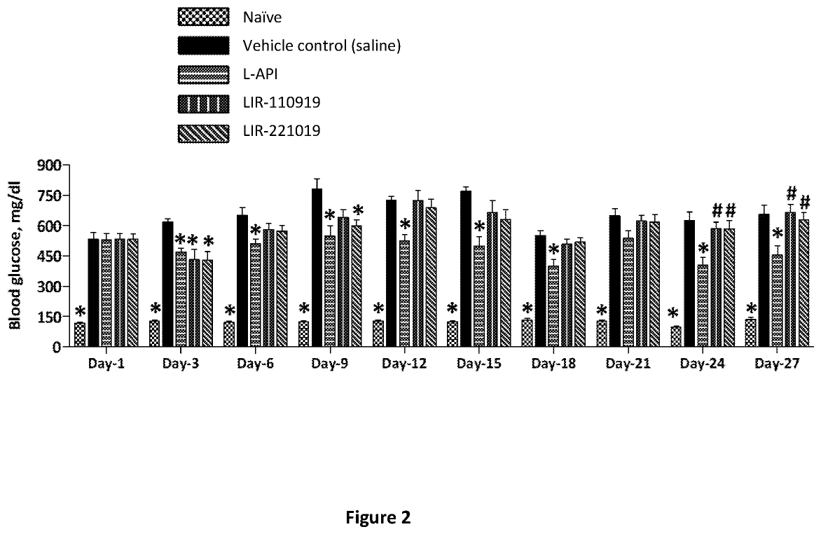

The effect of the test formulations on fed blood glucose levels is shown in FIG. 2. db/db mice showed statistically significant higher levels of fed blood glucose as compared to normal mice. L-API at 200 μg/kg, SC dose, administered daily showed significant reduction in ad-lib fed blood glucose levels post 3 hr. of treatment as compared to vehicle control. LIR-110919 (PLGA-liraglutide depot) at 156.3 mg/kg, IM dose, administered on Day-1 only, showed significant reduction in ad-lib fed blood glucose levels on Day-3 as compared to vehicle control. LIR-221019 (PCL-PLA/PLGA liraglutide depot) at 266.7 mg/kg, IM dose, administered on Day-1 only, showed significant reduction in ad-lib fed blood glucose levels on Day-3 and Day-9 as compared to vehicle control. The results are summarized in Table 2.

TABLE 2

| | Day 3 | Day 3 | Day 9 | Day 9 |
|---|---|---|---|---|
| | Mean ± SEM | % change vs. | mean ± SEM | % change vs. |
| Group | (mg/dL) | vehicle control | (mg/dL) | vehicle control |
| Control | 127.2 ± 4.8 | — | 123.5 ± 7.4 | — |
| (Naïve) | | | | |
| db/db vehicle | 615.4 ± 17.0 | — | 780.8 ± 51.4 | — |
| control | | | | |
| L-API | 468.3 ± 18.6 | −23.9% | 547.4 ± 49.9 | −29.9% |
| LIR-110919 | 431.0 ± 49.6 | −30.0% | 640.8 ± 36.0 | −17.9% |
| (PLGA- | | | | |
| liraglutide | | | | |
| depot) | | | | |
| LIR-221019 | 428.1 ± 41.9 | −30.4% | 598.9 ± 28.6 | −23.3% |
| (PCL-PLA/ | | | | |
| PLGA | | | | |
| liraglutide | | | | |
| depot) | | | | |

Effect of Test Formulations on HbA1C in db/db Mice

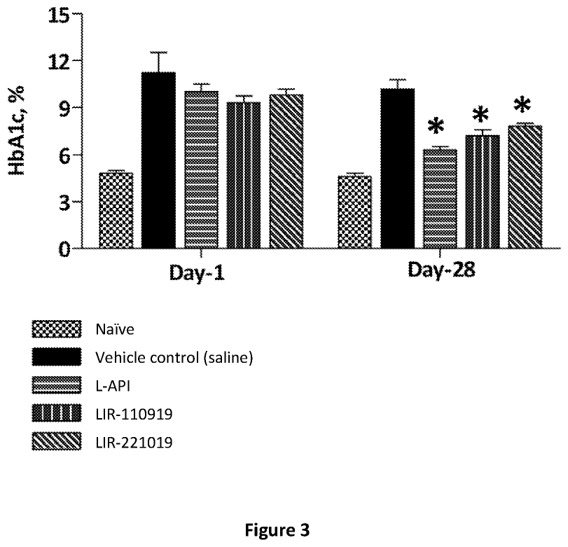

The effect of the test formulations on HbA1C levels is shown in FIG. 3. db/db mice showed statistically significant higher levels of HbA1c as compared to normal mice. L-API at 200 μg/kg, SC dose, administered daily showed significant reduction in HbA1C levels on Day-28 as compared to vehicle control. LIR-110919 (PLGA-liraglutide depot) at 156.3 mg/kg, IM dose administered on Day-1 only, showed significant reduction in HbA1C levels on Day-28 as compared to vehicle control. LIR-221019 (PCL-PLA/PLGA liraglutide depot) at 266.7 mg/kg, IM dose administered on Day-1 only, showed significant reduction in HbA1C levels on Day-28 as compared to vehicle control. The results are summarized in Table 3.

TABLE 3

| | Day 1 | Day 1 | Day 28 | Day 28 |
|---|---|---|---|---|
| | Mean HbA1c | % change vs. | mean HbA1c | % change vs. |
| Group | % ± SEM | vehicle control | % ± SEM | vehicle control |
| Control | 4.8 ± 0.1 | — | 4.6 ± 0.2 | — |
| (Naïve) | | | | |
| db/db vehicle | 12.5 ± 1.7 | — | 10.2 ± 0.6 | — |
| control | | | | |
| L-API | 10.0 ± 0.5 | −20.0% | 6.3 ± 0.2 | −38.2% |
| LIR-110919 | 9.3 ± 0.4 | −25.6% | 7.2 ± 0.4 | −29.4% |
| (PLGA- | | | | |
| liraglutide | | | | |
| depot) | | | | |
| LIR-221019 | 9.8 ± 0.4 | −21.6% | 7.8 ± 0.2 | −23.5% |
| (PCL-PLA/ | | | | |
| PLGA | | | | |
| liraglutide | | | | |
| depot) | | | | |

Effect of Test Formulations on Body Weight in db/db Mice

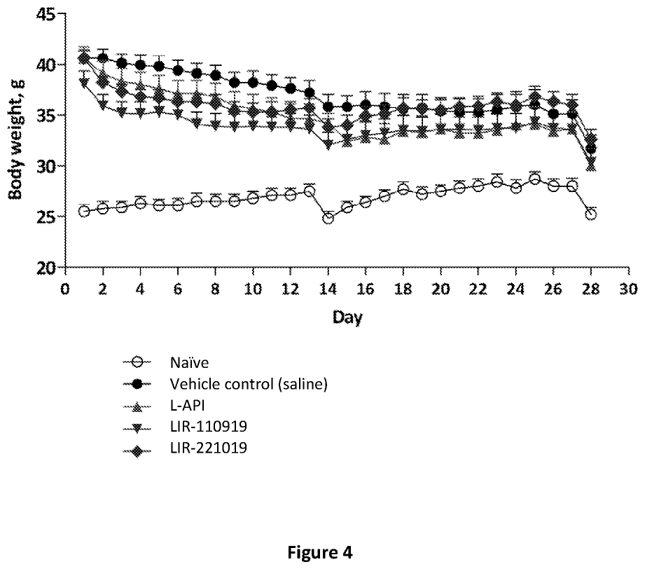

The effect of the test formulations on body weight is shown in FIG. 4. L-API at 200 μg/kg, SC dose, administered daily showed a trend towards reduction in body weight as compared to vehicle control. LIR-110919 (PLGA-liraglutide depot) at 156.3 mg/kg, IM dose, administered on Day-1 only, showed a trend towards reduction in body weight as compared to vehicle control. LIR-221019 (PCL-PLA/PLGA liraglutide depot) at 266.7 mg/kg, IM dose, administered on Day-1 only, showed a trend towards reduction in body weight as compared to vehicle control.

Plasma Concentrations of Liraglutide Formulations in db/db Mice

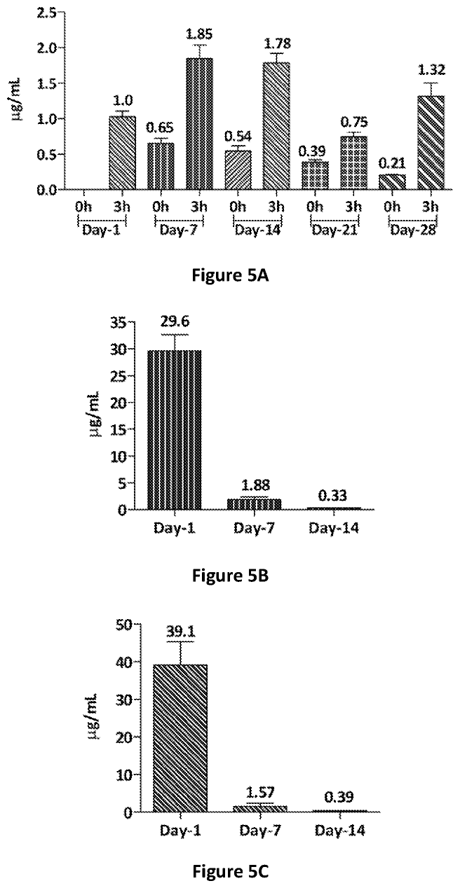

Plasma concentrations of liraglutide are shown in FIGS. 5A-5C. L-API at 200 μg/kg, SC dose, administered daily showed an increase in plasma levels post 3 hr. of injection and detectable plasma levels post 24 hr. of treatment. LIR-110919 (PLGA-liraglutide depot) at 156.3 mg/kg, IM and LIR-221019 (PCL-PLA/PLGA liraglutide depot) at 266.7 mg/kg, IM dose, administered on Day-1 only, showed declining plasma levels from Day-1 to Day-14. Plasma levels were not detectable on Day-21 and Day-28 for both formulations. Results are summarized in Tables 4A-4C.

TABLE 4A
L-API

| | | Plasma | Plasma Conc. nmol/L) |
|---|---|---|---|
| | | Conc. | (based on liraglutide |
| Day | Time | (μg/ml) | MW = 3751.2 g/mol) |
| 1 | 0 | BLQ | — |
| | 3 | 1.03 ± 0.08 | 274.6 |
| 7 | 0 | 0.65 ± 0.07 | 173.3 |
| | 3 | 1.85 ± 0.19 | 493.2 |
| 14 | 0 | 0.54 ± 0.07 | 144.0 |
| | 3 | 1.78 ± 0.14 | 474.5 |
| 21 | 0 | 0.39 ± 0.03 | 104.0 |
| | 3 | 0.75 ± 0.06 | 199.9 |
| 28 | 0 | 0.21 ± 0.01 | 56.0 |
| | 3 | 1.32 ± 0.18 | 351.9 |

BLQ ((below level of quantification) <0.178 mg/kg)

TABLE 4B
LIR-110919 (PLGA-liraglutide depot)

| | Plasma | Plasma Conc. nmol/L) |
|---|---|---|
| | Conc. | (based on liraglutide |
| Day | (μg/ml) | MW = 3751.2 g/mol) |
| 1 | 29.6 ± 3.0 | 7890.8 |
| 7 | 1.88 ± 0.48 | 501.2 |
| 14 | 0.33 ± 0.02 | 88.0 |

TABLE 4C
LIR-221019 (PCL-PLA/PLGA-liraglutide depot)

| | Plasma | Plasma Conc. nmol/L) |
|---|---|---|
| | Conc. | (based on liraglutide |
| Day | (μg/ml) | MW = 3751.2 g/mol) |
| 1 | 39.1 ± 6.3 | 10423.3 |
| 7 | 1.57 ± 0.82 | 418.5 |
| 14 | 0.39 ± 0.04 | 104.0 |

Thus, the depot compositions of the present invention maintain therapeutic plasma concentrations of liraglutide for at least 14 days after a single administration.

### Example 13: Comparison of Liraglutide Formulations in C57bl Mice Model

The objective of this study was to test the pharmacokinetic profile of various liraglutide depot formulations in male C57BL/6 mice with specific evaluation of Day-1 plasma concentration as an in vivo marker of the “burst” effect.

Treatment Groups: 1. Liraglutide API (L-API), 200 μg/kg, subcutaneous (SC), daily injections (n=3)
2. PLGA encapsulated liraglutide depot (depot 1—prepared according to Example 1, and depot LB1—low burst formulation, prepared according to Example 11A), dosed at 20 mg/kg (liraglutide based), intramuscular (IM) on Day-1 only (n=3 for each formulation)
3. PLGA/PCL-PLA encapsulated liraglutide depot (depot 2—prepared according to Example 2, and depot LB2—low burst formulation, prepared according to Example 11B), dosed at 20 mg/kg (liraglutide based), intramuscular (IM) on Day-1 only (n=3 for each formulation)

Total number of mice: 21

Formulation preparation: Depot formulations were suspended in a diluent containing carboxymethyl cellulose sodium (1%), polysorbate 20 (0.05%), sodium chloride (0.8%), and water for injection (98.1%). Liraglutide API was dissolved in water for injection.
Evaluated parameters: Liraglutide plasma concentration

### Results:

### Effect of Test Formulations on Liraglutide Plasma Concentration on Day-1 in Mice

Plasma concentrations of liraglutide in C57bl mice following administration of 20 mg/ml liraglutide based depot formulations are shown in Table 5.

TABLE 5

| Depot | Plasma concentration |
|---|---|
| Formulation | (ng/ml) Day 1 |
| 1 | 21805.5 |
| 2 | 46362.8 |
| LB1 | 9528.9 |
| LB2 | 14784.5 |

While the mean plasma concentrations of depots 1 and 2 a day after administration were comparable to the previously obtained results in db/db mice (Tables 4B and 4C), the low-burst formulations (depots LB1 and LB2) showed a 68-56% reduction in liraglutide plasma concentrations in the first 24 hours post administration.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1-45. (canceled)

46. A long-acting parenteral pharmaceutical composition comprising microparticles comprising dried water-in-oil-in-water (w/o/w) double emulsion droplets comprising an internal aqueous phase comprising a therapeutically effective amount of liraglutide or a pharmaceutically acceptable salt thereof; a water immiscible polymeric phase comprising a biodegradable carrier selected from the group consisting of polylactides, polyglycolides, polycaprolactones, and combinations thereof; and an external aqueous phase, wherein the composition is in depot form suitable for implantation at a medically acceptable location in a subject in need thereof at a frequency of once weekly to once every six months and provides a twenty-four hour liraglutide burst release of less than 15% of the administered dose following administration.

47. The long-acting parenteral pharmaceutical composition of claim 46, wherein the biodegradable carrier is a polymer selected from the group consisting of poly (D,L-lactide-co-glycolide) (PLGA), poly (D,L-lactide) (PLA), polyglycolide (PGA), polycaprolactone (PCL), and combinations thereof.

48. The long-acting parenteral pharmaceutical composition of claim 47, wherein the biodegradable carrier is poly (D,L-lactide-co-glycolide) (PLGA); or wherein the biodegradable carrier is poly (D,L-lactide) (PLA); or wherein the biodegradable carrier is PLA-PCL; or wherein the biodegradable carrier is a mixture of PLA-PCL and PLGA.

49. The long-acting parenteral pharmaceutical composition of claim 46, wherein the liraglutide is present in the composition in the form of a free base.

50. The long-acting parenteral pharmaceutical composition of claim 46, wherein the composition releases the liraglutide active ingredient over a period of about one week to about three months; or wherein the composition releases the liraglutide active ingredient over a period of about one week to about one month; or wherein the composition releases the liraglutide active ingredient over a period of about one week to about two weeks.

51. A method of treating type-2 diabetes mellitus, comprising the step of administering to a subject in need thereof the long-acting parenteral pharmaceutical composition of claim 46 at a frequency of once weekly to once every six months.

52. The method of claim 51, wherein treating type-2 diabetes mellitus comprises reducing fasting glucose levels in a subject by at least about 20% for a time period between about 1 week and about 28 days after a single administration; or wherein treating type-2 diabetes mellitus comprises reducing fed glucose levels in a subject by at least about 10% for a time period between about 1 week and about 28 days after a single administration; or wherein treating type-2 diabetes mellitus comprises reducing hemoglobin A1C (HbA1C) levels in a subject by at least about 20% for a time period between about 1 week and about 28 days after a single administration.

53. A method of treating Parkinson's Disease, comprising the step of administering to a subject in need thereof the long-acting parenteral pharmaceutical composition of claim 46 at a frequency of once weekly to once every six months.

54. A long-acting parenteral pharmaceutical composition comprising microparticles comprising dried water-in-oil-in-water (w/o/w) double emulsion droplets comprising an internal aqueous phase comprising a therapeutically effective amount of liraglutide or a pharmaceutically acceptable salt thereof; a water immiscible polymeric phase comprising a biodegradable carrier selected from the group consisting of polylactides, polyglycolides, polycaprolactones, and combinations thereof; and an external aqueous phase, wherein the composition is in depot form suitable for implantation at a medically acceptable location in a subject in need thereof at a frequency of once weekly to once every six months and provides average human steady-state plasma concentrations (C_{ss,avg}) of liraglutide of at least about 5 nmol/L for about 1 week to about 28 days after a single administration.

55. The long-acting parenteral pharmaceutical composition of claim 54, wherein the average human steady-state plasma concentrations of liraglutide are between about 5 and about 45 nmol/L.

56. The long-acting parenteral pharmaceutical composition of claim 54, wherein the biodegradable carrier is a polymer selected from the group consisting of poly (D,L-lactide-co-glycolide) (PLGA), poly (D,L-lactide) (PLA), polyglycolide (PGA), polycaprolactone (PCL), and combinations thereof.

57. The long-acting parenteral pharmaceutical composition of claim 56, wherein the biodegradable carrier is poly (D,L-lactide-co-glycolide) (PLGA); or wherein the biodegradable carrier is poly (D,L-lactide) (PLA); or wherein the biodegradable carrier is PLA-PCL; or wherein the biodegradable carrier is a mixture of PLA-PCL and PLGA.

58. The long-acting parenteral pharmaceutical composition of claim 54, wherein the liraglutide is present in the composition in the form of a free base.

59. The long-acting parenteral pharmaceutical composition of claim 54, wherein the composition releases the liraglutide active ingredient over a period of about one week to about three months; or wherein the composition releases the liraglutide active ingredient over a period of about one week to about one month; or wherein the composition releases the liraglutide active ingredient over a period of about one week to about two weeks.

60. A method of treating type-2 diabetes mellitus, comprising the step of administering to a subject in need thereof the long-acting parenteral pharmaceutical composition of claim 54 at a frequency of once weekly to once every six months.

61. The method of claim 60, wherein treating type-2 diabetes mellitus comprises reducing fasting glucose levels in a subject by at least about 20% for a time period between about 1 week and about 28 days after a single administration; or wherein treating type-2 diabetes mellitus comprises reducing fed glucose levels in a subject by at least about 10% for a time period between about 1 week and about 28 days after a single administration; or wherein treating type-2 diabetes mellitus comprises reducing hemoglobin A1C (HbA1C) levels in a subject by at least about 20% for a time period between about 1 week and about 28 days after a single administration.

62. A method of treating Parkinson's Disease, comprising the step of administering to a subject in need thereof the long-acting parenteral pharmaceutical composition of claim 54 at a frequency of once weekly to once every six months.

63. A method of achieving average human steady-state plasma concentrations (C_{ss,avg}) of liraglutide of at least about 5 nmol/L for about 1 week to about 28 days after a single administration, the method comprising the step of administering to a subject in need thereof a long-acting parenteral pharmaceutical composition comprising liraglutide or a pharmaceutically acceptable salt thereof and a biodegradable carrier selected from the group consisting of polylactides, polyglycolides, polycaprolactones, and combinations thereof, wherein the composition is in depot form suitable for implantation at a medically acceptable location in a subject in need thereof.

64. The method of claim 63, wherein the average human steady-state plasma concentrations of liraglutide are between about 5 and about 45 nmol/L; or wherein the liraglutide depot formulation is administered at a dose between about 5 mg and about 100 mg of liraglutide.

65. The method of claim 63, wherein the liraglutide is present in the long-acting parenteral pharmaceutical composition in the form of a free base; or wherein the biodegradable carrier is selected from the group consisting of poly (D,L-lactide-co-glycolide) (PLGA), poly (D,L-lactide) (PLA), polyglycolide (PGA), polycaprolactone (PCL), and combinations thereof.

66. The method of claim 65, wherein the biodegradable carrier is poly (D,L-lactide-co-glycolide) (PLGA); or wherein the biodegradable carrier is poly (D,L-lactide) (PLA); or wherein the biodegradable carrier is PLA-PCL; or wherein the biodegradable carrier is a mixture of PLA-PCL and PLGA.

67. The method of claim 63, wherein the composition comprises microparticles comprising dried water-in-oil-in-water (w/o/w) double emulsion droplets comprising an internal aqueous phase comprising a therapeutically effective amount of liraglutide or a pharmaceutically acceptable salt thereof; a water immiscible polymeric phase comprising a biodegradable carrier selected from the group consisting of polylactides, polyglycolides, polycaprolactones, and combinations thereof; and an external aqueous phase.
